(19) United States
(12) Patent Application Publication
(10) Pub. No.: US 2024/0351044 A1
(43) Pub. Date: Oct. 24, 2024

(54) SYSTEM AND METHOD TO PERFORM QUANTITATIVE POLYMERASE CHAIN REACTION

(71) Applicant: IXENSOR CO., LTD., Taipei (TW)

(72) Inventors: Jiun-Yann YU, Taipei (TW); Yenyu CHEN, Taipei (TW)

(73) Assignee: IXENSOR CO., LTD., Taipei (TW)

(21) Appl. No.: 18/577,265

(22) PCT Filed: Jul. 8, 2022
(86) PCT No.: PCT/CN2022/104624

§ 371 Date: Jan. 7, 2024

Related U.S. Application Data
(60) Provisional application No. 63/219,810, filed on Jul. 8, 2021.

(51) Int. Cl. B01L 7/00; C12Q 1/6851; G01N 21/64

(52) U.S. Cl. CPC B01L 7/52; C12Q 1/6851; G01N 21/6428; B01L 2200/0647; B01L 2300/1861; B01L 2300/1894; G01N 2021/6439; G01N 2201/0636

(57) ABSTRACT

Example methods and systems for performing quantitative polymerase chain reaction (qPCR) are provided. One example system may include a first lighting subsystem and a second lighting subsystem. The first lighting subsystem includes a first light source configured to illuminate a qPCR testing solution including a sample, a fluorescence dye and a nanoparticle in a tube with a first light to increase a temperature of the qPCR testing solution. The second lighting subsystem includes a light detector configured to detect an amount of fluorescence emitted by the fluorescence dye in a first thermal cycle of the qPCR. The first lighting subsystem is configured to turn off the first light source for a period of time in any thermal cycle of the qPCR after the first thermal cycle based on one or more heat transmission parameters which are determined based on the amount of fluorescence.

### CROSS-REFERENCE TO RELATED APPLICATIONS

The present application is a U.S. national stage filing under 35 U.S. C. § 371 of International Application No. PCT/CN2022/104624, filed Jul. 8, 2022, which claims the benefit of U.S. Provisional Application No. 63/219,810, filed Jul. 8, 2021. The International Application and the U.S. Provisional Application above are incorporated by reference in their entirety.

### BACKGROUND

Quantitative polymerase chain reaction (qPCR) is an operation by which an amount of a deoxyribonucleic acid (DNA) target region can be determined, in real-time, and is widely applied in detections and quantifications of various microbial agents.

Conventional qPCR is performed by a thermal cycler. The thermal cycler includes a thermal block with one or more holes. Each of the holes is configured to hold a tube including a sample collected from a patient.

To perform qPCR, the thermal cycler is configured to heat the tube to a higher predefined temperature (e.g., around 90 to 99 degrees Celsius) for a first period of time by heating the thermal block to the higher predefined temperature and then cool the tube to a lower predefined temperature (e.g., around 50 to 70 degrees Celsius) for a second period of time by cooling the thermal block to the lower predefined temperature. A cycle of increasing the temperature of the tube to the higher predefined temperature for the first period of time and then lowering the temperature of the tube to the lower predefined temperature for the second period of time is referred to as a thermal cycle.

However, heating and cooling the thermal block require a significant amount of time. For example, it usually takes around four hours to complete forty thermal cycles of qPCR. There has been a long felt need for completing qPCR in a significantly reduced time period.

### SUMMARY

In examples of the present disclosure, systems configured to perform qPCR are provided. The systems may include a first lighting subsystem and a second lighting subsystem. The first lighting subsystem includes a first light source configured to illuminate a qPCR testing solution including a sample, a fluorescence dye and a nanoparticle in a tube with a first light to increase a temperature of the qPCR testing solution. The second lighting subsystem includes a light detector configured to detect an amount of fluorescence emitted by the fluorescence dye in a first thermal cycle of the qPCR. The first lighting subsystem is configured to turn off the first light source for a period of time in any thermal cycle of the qPCR after the first thermal cycle based on one or more heat transmission parameters which are determined based on the amount of fluorescence.

In examples of the present disclosure, methods to perform qPCR are provided. In a first thermal cycle of the qPCR, the methods may include determining one or more heat transmission parameters based on an amount of fluorescence emitted by a fluorescence dye included in a qPCR testing solution in a tube; and determining a first time duration of turning on a first light source configured to illuminate the qPCR testing solution further including a nanoparticle and a sample with a first light, a second time duration of turning off the first light source, a first power of the first light source and a second power of the first light source based on the one or more heat transmission parameters.

### BRIEF DESCRIPTION OF THE DRAWINGS

The foregoing and other features of the present disclosure will become more fully apparent from the following description and appended claims, taken in conjunction with the accompanying drawings. These drawings depict only several embodiments in accordance with the disclosure and are therefore not to be considered limiting of its scope. The disclosure will be described with additional specificity and detail through use of the accompanying drawings.

FIG. 1 illustrates a perspective view of system 100 configured to perform qPCR, according to some embodiments of the present disclosure.

FIG. 2 is a flow chart of method 200 for performing qPCR, according to some embodiments of the present disclosure.

FIG. 3 is a diagram showing relationships 300 among time, temperature, thermal cycles and detected amount of fluorescence, according to some embodiments of the present disclosure.

FIG. 4 is a diagram showing relationships 400 among time, temperature, thermal cycles and detected amount of fluorescence, according to some embodiments of the present disclosure.

FIG. 5 is an illustration of computing device 500 configured to perform various embodiments of the present disclosure.

FIG. 6 is a block diagram of an illustrative embodiment of a computer program product 600 for implementing various embodiments of the present disclosure

### DETAILED DESCRIPTION

In the following detailed description, reference is made to the accompanying drawings, which form a part hereof. In the drawings, similar symbols typically identify similar components and same numerals typically identify same components, unless context dictates otherwise. The illustrative embodiments described in the detailed description and drawings are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated herein.

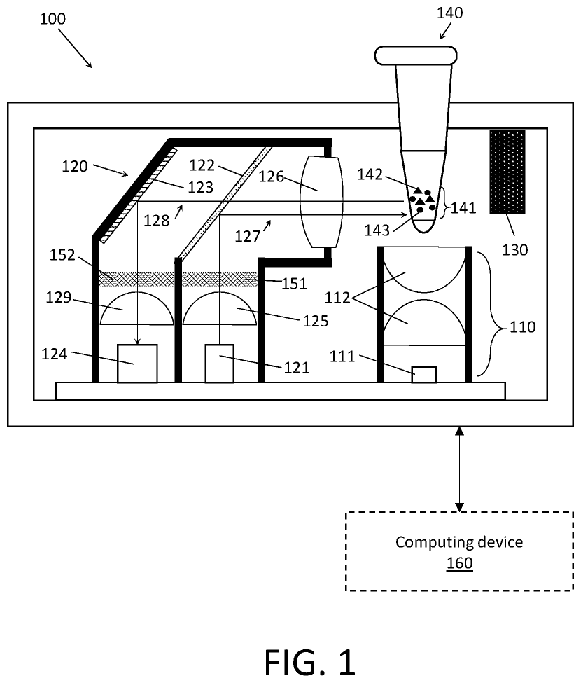

FIG. 1 illustrates a perspective view of system 100 configured to perform qPCR, according to some embodiments of the present disclosure. System 100 includes first lighting subsystem 110 and second lighting subsystem 120. In addition, system 100 is also configured to receive tube 140. Tube 140 is configured to hold qPCR testing solution 141, sample 142 collected from a patient and nanoparticles 143. Sample 142 and nanoparticles 143 are dispersed in qPCR testing solution 141. Some examples of nanoparticles 143 may be metal, such as gold, silver, etc. System 100 may be coupled to computing device 160, which may be external to system 100 or as part of system 100. Computing device 160 is configured with executable instructions for receiving and processing information from system 100 and issuing instructions to system 100. Some examples of computing device 160 may be, without limitation, an embedded system, a mobile device, a computer system, etc.

In some embodiments, sample 142 includes double-stranded deoxyribonucleic acid (DNA) sequences of the patient. For a patient who is infected by a particular virus (e.g., coronavirus), the DNA sequences includes a double-stranded DNA target region associated with the virus. In contrast, for a patient who is not infected by the virus, the DNA sequences do not include such double-stranded DNA target region. The double-stranded DNA target region includes a first single-stranded DNA sequence and a second single-stranded DNA sequence. The first single-stranded DNA sequence and the second single-stranded DNA sequence coil around each other.

In some embodiments, qPCR testing solution 141 includes a first DNA primer and a second DNA primer (the primers are not explicitly shown in FIG. 1). The first DNA primer is a single-stranded DNA sequence that is complementary to an end of the first single-stranded DNA sequence of the double-stranded DNA target region. Similarly, the second DNA primer is another single-stranded DNA sequence that is complementary to an end of the second single-stranded DNA sequence of the double-stranded DNA target region.

In some embodiments, first lighting subsystem 110 includes first light source 111. First light source 111 is configured to illuminate nanoparticles 143 to induce generation of plasmonic energy on nanoparticles 143 so that the plasmonic energy heats qPCR testing solution 141 and sample 142 to a higher predefined temperature (e.g., around 90 to 99 degrees Celsius) for a first period of time. At the higher predefined temperature, the double-stranded DNA target region is denaturized to form the first single-stranded DNA sequence of the double-stranded DNA target region and the second single-stranded DNA sequence of the double-stranded DNA target region. In some embodiments, first light source 111 is an infrared light source, such as an infrared light emitting diode.

In some embodiments, first lighting subsystem 110 optionally includes lens 112. Lens 112 is configured to facilitate focusing the lights emitted by first light source 111 onto nanoparticles 143.

In some embodiments, system 100 may include an optional cooling system 130. After the first period of time, cooling system 130 is configured to cool qPCR testing solution 141, sample 142 and nanoparticles 143 to a lower predefined temperature (e.g., around 50 to 70 degrees Celsius) for a second period of time. The cooling system 130 may include a fan. At the lower predefined temperature, the first primer and the second primer may couple to the ends of the first and second single-stranded DNA sequences of the double-stranded DNA target region, respectively. Alternatively, qPCR testing solution 141, sample 142 and nanoparticles 143 may be cooled to the lower predefined temperature naturally without using cooling system 130.

In some embodiments, qPCR testing solution 141 further includes DNA building blocks that are configured to extend single-stranded DNA sequences from the first and second primers at the lowered temperature, respectively. Accordingly, two new double-stranded DNA sequences including the DNA target region are formed. The number of the DNA target region is doubled in this thermal cycle (i.e., from 1 to 2).

In some embodiments, qPCR testing solution 141 further includes a fluorescence dye configured to couple to the double-stranded DNA target region. The fluorescence dye may be included in the primers. Therefore, in the scenario that the primers are capable of coupling to the single-stranded DNA sequences of the double-stranded DNA target region, which corresponds to the sample being from a patient who is infected by the virus, the numbers of the double-stranded DNA target regions, as well as the primers and the fluorescence dye included in the primers, will be exponentially doubled. Accordingly, an amount of fluorescence emitted by the fluorescence dye will also be increased.

In some embodiments, second lighting subsystem 120 includes second light source 121, dichroic mirror 122, reflector 123 and light detector 124. Second light source 121 is configured to generate and emit lights having a shorter wavelength (e.g., blue lights) than the wavelength of fluorescence. Second light source 121 may be a blue light emitting diode. The lights may be guided by dichroic mirror 122, optical elements 125 and 126 from second light source 121 to qPCR testing solution 141, sample 142 and nanoparticles 143 along first light path 127. Dichroic mirror 122 is configured to block the lights having the shorter wavelength from passing through but allow lights having a longer wavelength passing through. Optical elements 125 and 126 are optional and may include lens and/or light guides. First light path 127 may optionally include first color filter 151. First color filter 151 is configured to filter out lights having a wavelength of the fluorescence. For example, first color filter 151 may be configured to filter out green lights.

In some embodiments, the fluorescence dye included in qPCR testing solution 141 is configured to absorb the lights emitted by second light source 121 and emit an amount of fluorescence having the longer wavelength (e.g., green lights). The emitted fluorescence may travel along second light path 128. More specifically, the emitted fluorescence may be guided by optical element 126 to dichroic mirror 122. As discussed, dichroic mirror 122 is configured to allow the lights having the longer wavelength (e.g., emitted fluorescence) pass through. The emitted fluorescence is then reflected by reflector 123 through optical element 129 to light detector 124. Optical element 129 may be optional and include lens/light guides. Second light path 128 may also optionally include second color filter 152. Second color filter 152 is configured to filter out lights having a wavelength of second light source 121. For example, second color filter 152 may be configured to filter out blue lights.

In some embodiments, light detector 124 is configured to convert the amount of fluorescence into an electrical signal for further processing. For example, light detector 124 may be a photodiode. In some embodiments, higher amounts of fluorescence may result stronger electrical signals, which correspond to the exponentially increased numbers of the double-stranded DNA target region when the patient is infected by the virus.

Conventionally, as discussed above, the higher and lower predefined temperatures of qPCR testing solution 141 and sample 142 are precisely controlled by the thermal cycler. The thermal cycler is configured to control the temperature of the thermal block holding tube 140 to heat and cool qPCR testing solution 141 and sample 142 to the higher and lower predefined temperatures.

It should be noted that system 100 does not include a conventional thermal block. Instead, system 100 is configured to use the plasmonic energy generated by nanoparticles 143 to heat qPCR testing solution 141 and sample 142. qPCR testing solution 141 and sample 142 may be naturally cooled without cooling system 130 or cooled by cooling system 130. Embodiments of system 100 supports an innovative approach to control the temperature of qPCR testing solution 141 and sample 142.

In some embodiments, the temperature of qPCR testing solution 141 and sample 142 may be controlled by a time duration of turning on first light source 111 to increase the temperature, adjusting the power of first light source 111 to a first power so that the temperature is maintained at the higher predefined temperature, a time duration of turning off first light source 111 to decrease the temperature and adjusting the power of first light source 111 to a second power so that the temperature is maintained at the lower predefined temperature. The time durations and the adjusted powers may be determined based on a relationship between an amount of fluorescence emitted by the fluorescence dye in qPCR testing solution 141 and an estimated temperature of sample 142 in the first thermal cycle of qPCR. The details of determining the time durations and powers are described below.

In general, a temperature change rate of a qPCR sample can be described as

$$\frac{d T}{dt} = - α ( T - {T}_{Env} ) + P , \qquad ( 1 )$$

where T is the estimated temperature of sample 142, t is time, α is the heat conduction coefficient determined by sample 142 and the environment of sample 142, T_{Env} is the environment temperature, and P is the power of an internal heat source (e.g., nanoparticles 143). More specifically, P can be estimated as a product of a light source power, P_{Light} (e.g., power of first light source 111) and a light-heat conversion coefficient β.

In some embodiments, Eq. (1) is a first-order ordinary differential equation, the estimated temperature T of sample 142 at any t may be obtained as long as the initial condition (i.e., the temperature of sample 142 at t=0) is given. In addition, based on Eq. (1), T_{Env}, P_{Light} and a time duration to reach a certain estimated temperature T may be also calculated. Nonetheless, heat transmission parameters, such as the heat conduction coefficient α and the light-heat conversion coefficient f are usually unknown, and they likely vary from one sample to another.

In some embodiments, values of α and β may be obtained by measuring how the amount of fluorescence emitted by the fluorescence dye in qPCR testing solution 141 vary with time in the first thermal cycle of qPCR. For example, in the first thermal cycle of qPCR, qPCR testing solution and sample 142 are heated from a room temperature of 15 to 25 degrees Celsius to 90 degrees Celsius. At a temperature range of 15 to 35 degrees Celsius, the fluorescence dye (e.g., SYBR Green) included in qPCR testing solution 141 is observed to emit significant amounts of fluorescence. Moreover, a relationship between an amount of fluorescence emitted by the fluorescence dye at a corresponding temperature is highly consistent from sample to sample. Throughout the present disclosure, this relationship is referred to as a “fluorescence-temperature relation.”

Although the temperature range of 15 to 35 degrees Celsius is not within the temperature range to perform the qPCR (i.e., temperatures between the higher predefined temperature, for example 90 degrees Celsius, and the lower predefined temperature, for example 60 degrees Celsius), the fluorescence-temperature relation in the temperature range of 15 to 35 degrees Celsius is sufficient to determine the values of α and β above.

In some embodiments, Eq. (1) may be rewritten to

$$\frac{d T}{dt} = - α ( T - {T}_{Env} ) + β {P}_{Light} \qquad ( 2 )$$

In some embodiments, T is the estimated temperature of sample 142, t is time, T_{Env} is the environment temperature, and P_{Light} is light source power of first light source 111. By turning on first light source 111 to heat sample 142 at various time points and record temperatures at the various time points, a plot of d T/dt against T−T_{En}v may be obtained based on the time points and corresponding temperature at the time points. Based on Eq. (2), the tangent of a linear regression of the plot is −α, and its intersection with the y-axis is βP_{Light}. As such, values of α and β may be obtained when P_{Light} is known.

With α and β obtained, information needed for calculating the temperature T as a function of t is sufficient. Given that α, β, T_{Env}, and P_{Light} are all known constants now, Eq. (2) is a first-order ordinary differential equation that has a solution of:

$$T ( t ) = {T}_{Env} + \frac{β {P}_{L i g h t}}{α} + A {e}^{- α t} \qquad ( 3 )$$

where A is a constant to be determined by an initial condition. Assuming that the estimated temperature of sample 142 at t=0 is the environment temperature, T_{Env}, A then equals to −βP_{Light}/α, and Eq. (3) may be rewritten to:

$$T ( t ) = {T}_{Env} + \frac{β {P}_{Light}}{α} ( 1 - {e}^{- α t} ) \qquad ( 4 )$$

In some embodiments, based on actual measurements of temperatures of sample 142 by a thermometer, the mean square difference between the estimated temperature T(t) and the actual measured temperature is only around 1 to 2 degrees of Celsius. This difference is acceptable to measure the higher predefined temperature and the lower predefined temperature to perform qPCR. Therefore, the estimated temperature T(t) calculated based on equations above may be treated as the temperature of sample 142 in a qPCR.

In some embodiments, based on Eq. (4), the time duration of turning on first light source 111 for sample 142 to reach a certain temperature (e.g., the higher predefined temperature) may be derived from an inverse function Eq. (5) of Eq. (4):

$${t}_{Heating} ( T ) = - \frac{1}{α} ln ( 1 - \frac{α}{β {P}_{Light}} ( T - {T}_{Env} ) ) \qquad ( 5 )$$

In some embodiments, Eq. (6) may be derived from Eq. (5). Eq. (6) may calculate a time duration of turning on first light source 111 for sample 142 to increase its temperature from an arbitrary temperature, Tₐ (e.g., the lower predefined temperature, for example 60 degrees Celsius), to another arbitrary temperature, T_{b} (e.g., the higher predefined temperature, for example 90 degrees Celsius), as:

$${t}_{Heating} ( {T}_{a} \to {T}_{b} ) = t ( {T}_{b} ) - t ( {T}_{a} ) = \frac{1}{α} ln ( \frac{\frac{β {P}_{Light}}{α} + {T}_{Env - {T}_{a}}}{\frac{β {P}_{Light}}{α} + {T}_{Env} - {T}_{b}} ) \qquad ( 6 )$$

Similarly, if the temperature of sample 142 is to be dropped from T_{b} to Tₐ, first light source 111 (i.e., P_{Light}=0) can be turned off and remains turned off for a time duration, as shown in Eq. (7) below.

$${t}_{Cooling} ( {T}_{b} \to {T}_{a} ) = \frac{1}{α} ln ( \frac{{T}_{Env - {T}_{b}}}{{T}_{Env - {T}_{a}}} ) \qquad ( 7 )$$

In some other embodiments, a power to hold sample 142 at a temperature can be derived once that temperature is reached by simply making Eq. (2) equal to 0, as:

$$\frac{d T}{d t} = - α ( T - {T}_{Env} ) + β {P}_{Light} = 0$$

Then the power of first light source 111 to hold sample 142 at the temperature is:

$${P}_{Light} ( T ) = \frac{α}{β} ( T - {T}_{Env} ) \qquad ( 8 )$$

Therefore, the power of first light source 111 to hold sample 142 at the higher predefined temperature is

$${P}_{Light} ( {T}_{b} ) = \frac{α}{β} ( {T}_{b} - {T}_{Env} )$$

and the power of first light source 111 to hold sample 142 at the lower predefined temperature is

$${P}_{Light} ( {T}_{a} ) = \frac{α}{β} ( {T}_{a} - {T}_{Env} ) .$$

In some embodiments, any thermal cycle of the qPCR after the first thermal cycle can be completed by heating sample 142 for a period of time t_{Heating}(Tₐ→T_{b}) so that temperature of sample 142 may be increased from the lower predefined temperature of 60 degrees Celsius to the higher predefined temperature of 90 degrees Celsius, keeping sample 142 at the higher predefined temperature by adjusting the power of first light source 111 based on P_{Light}(T_{b}), cooling sample 142 for a period of time t_{cooling}(T_{b}→Tₐ) so that temperature of sample 142 may be decreased from the higher predefined temperature to the lower predefined temperature and keeping sample 142 at the lower predefined temperature by adjusting the power of first light source 111 based on P_{Light}(Tₐ).

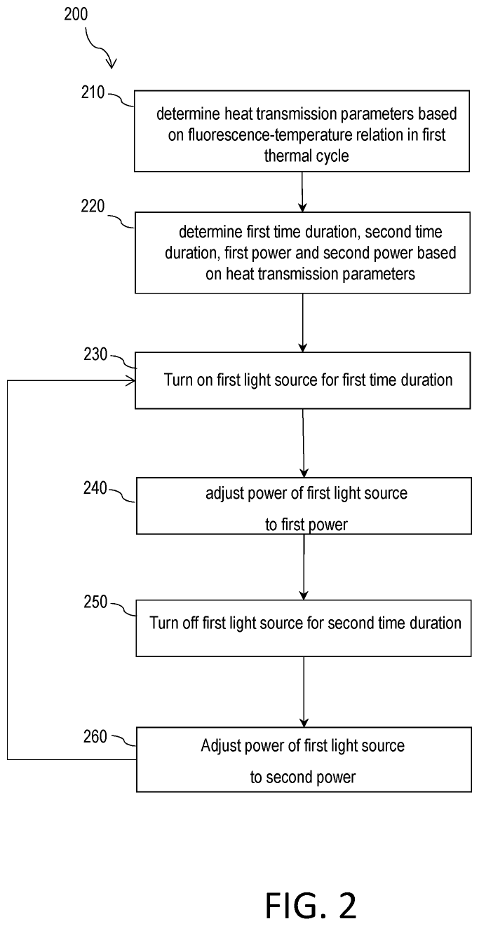

FIG. 2 is a flowchart of method 200 for performing qPCR, according to some embodiments of the present disclosure. Method 200 may include one or more operations, functions, or actions illustrated by one or more blocks. Although the blocks of method 200 and other methods described herein are illustrated in sequential orders, these blocks may also be performed in parallel, or in a different order than those described herein. Also, the various blocks may be combined into fewer blocks, divided into additional blocks, or eliminated based upon the desired implementation. Method 200 may begin in block 210.

In some embodiments, in block 210 “determine heat transmission parameters based on fluorescence-temperature relation in first thermal cycle”, as discussed above and in conjunction with FIG. 1, in the first thermal cycle based on Eq. (1) and (2), computing device 160 is configured to determine heat transmission parameters a and β. Block 210 may be followed by block 220.

In some embodiments, in block 220 “determine first time duration, second time duration, first power and second power based on heat transmission parameters”, as discussed above and in conjunction with FIG. 1, computing device 160 is configured to determine a first time duration of turning on first light source 111 for qPCR testing solution 141 and sample 142 to increase their temperatures from an arbitrary temperature, Tₐ (e.g., the lower predefined temperature, for example 60 degrees Celsius), to another arbitrary temperature, T_{b} (e.g., the higher predefined temperature, for example 90 degrees Celsius) based on Eq. (6). Similarly, computing device 160 is configured to determine a second time duration of turning off first light source 111 for qPCR testing solution 141 and sample 142 to decrease their temperatures from the arbitrary temperature, T_{b} (e.g., the higher predefined temperature, for example 90 degrees Celsius), to another arbitrary temperature, Tₐ (e.g., the lower predefined temperature, for example 60 degrees Celsius) based on Eq. (7). In addition, computing device 160 is configured to determine a first power of first light source 111 to hold qPCR testing solution 141 and sample 142 at the higher predefined temperature and a second power of first light source 111 to hold qPCR testing solution 141 and sample 142 at the lower predefined temperature based on Eq. (8). In some embodiments, blocks 210 and 220 are carried out in the first thermal cycle of qPCR having multiple thermal cycles (e.g., forty thermal cycles). Block 220 may be followed by block 230.

In some embodiments, in block 230 “turn on first light source for first time duration”, in conjunction with FIG. 1, for any thermal cycle after the first thermal cycle (e.g., the second thermal cycle), computing device 160 is configured to turn on first light source 111 for the first time duration determined in block 220. Therefore, the temperature of qPCR testing solution 141 and sample 142 may be increased from an arbitrary temperature, Tₐ (e.g., the lower predefined temperature, for example 60 degrees Celsius), to another arbitrary temperature, T_{b} (e.g., the higher predefined temperature, for example 90 degrees Celsius). Block 230 may be followed by block 240.

In some embodiments, in block 240 “adjust power of first light source to first power”, in conjunction with FIG. 1, for any thermal cycle after the first thermal cycle (e.g., the second thermal cycle), computing device 160 is configured to adjust the power of first light source 111 to the first power determined in block 220. In some embodiments, computing device 160 is configured to apply certain amount of current to first light source 111, resulting in the first power (e.g., in milliwatts (mW)). Therefore, the temperature of qPCR testing solution 141 and sample 142 may be kept at T_{b} (e.g., the higher predefined temperature, for example 90 degrees Celsius). Block 240 may be followed by block 250.

In some embodiments, in block 250 “turn off first light source for second time duration”, in conjunction with FIG. 1, for any thermal cycle after the first thermal cycle (e.g., the second thermal cycle), computing device 160 is configured to turn off first light source 111 for the second time duration determined in block 220. This turning off of first light source 111 may cause the temperature of qPCR testing solution 141 and sample 142 to be decreased from T_{b} to Tₐ. Block 250 may be followed by block 260.

In some embodiments, in block 260 “adjust power of first light source to second power”, in conjunction with FIG. 1, for any thermal cycle after the first thermal cycle (e.g., the second thermal cycle), computing device 160 is configured to adjust the power of first light source 111 to the second power determined in block 220. In some embodiments, computing device 160 is configured to apply another amount of current to first light source 111, resulting in the second power (e.g., in milliwatts (mW)). This adjustment of first light source 111's power may maintain the temperature of qPCR testing solution 141 and sample 142 at Tₐ. Blocks 230, 240, 250 and 260 may be performed to complete a thermal cycle of qPCR after the first thermal cycle. In some embodiments, block 260 may be looped back to block 230 for a next thermal cycle of qPCR. Blocks 230, 240, 250 and 260 may be repeated for a required number of thermal cycles to perform qPCR (e.g., 39 thermal cycles). Unlike conventional thermal block approaches, performing a 40-thermal-cycle (i.e., the first thermal cycle corresponding to blocks 210 and 220 and 39 thermal cycles corresponding to blocks 230, 240, 250 and 260) qPCR according to some embodiments of the present disclosure can take as little as 10 to 20 minutes.

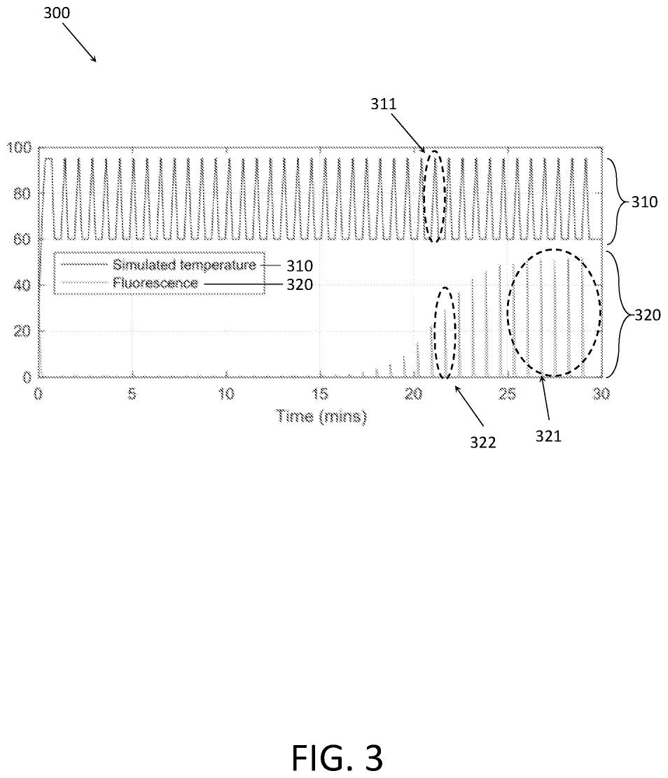

FIG. 3 is a diagram showing relationships 300 among time, temperature, thermal cycles and detected amount of fluorescence, according to some embodiments of the present disclosure.

In some embodiments, relationships 300 may include first curve 310 and second curve 320. In conjunction with FIG. 1, first curve 310 represents a relationship between an estimated temperature of sample 142 and a duration of time in which qPCR is performed. In some embodiments, first curves 310 include multiple peaks 311. Each peak 311 represents a thermal cycle.

In some embodiments, second curve 320 represents a relationship between an amount of fluorescence and the duration of time in which qPCR is performed.

Second curves 320 includes various peaks representing the amount of fluorescence. In conjunction with FIG. 1, the change of amounts of fluorescence in second curve 320 suggests the number of the DNA target region is exponentially doubled and sample 142 is collected from a patient who is infected by the virus. Second curve 320 includes peaks 321 showing the maximum amount of fluorescence. In some embodiments, peak 322 in the middle of the last peak showing the minimum amount of fluorescence and the first peak showing the maximum amount of fluorescence may be determined as a point that a qPCR reaction rate of sample 142 reaches a threshold. The thermal cycle peak 311 corresponding to this peak 322 may be referred to as threshold cycle (Ct).

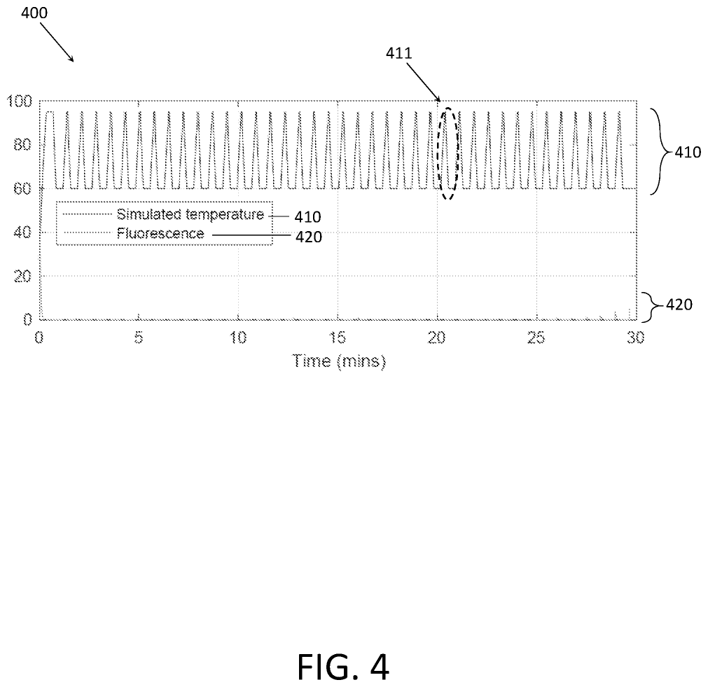

FIG. 4 is a diagram showing relationships 400 among time, temperature, thermal cycles and detected amount of fluorescence, according to some embodiments of the present disclosure.

In some embodiments, relationships 400 may include first curve 410 and second curve 420. In conjunction with FIG. 1, first curve 410 represents a relationship between an estimated temperature of sample 142 and a duration of time in which qPCR is performed. In some embodiments, first curves 410 include multiple peaks 411. Each peak 411 represents a thermal cycle.

In some embodiments, second curve 420 represents a relationship between an amount of fluorescence and the duration of time in which qPCR is performed.

Second curves 420 includes various peaks representing the amount of fluorescence. In conjunction with FIG. 1, the very few change of amounts of fluorescence in second curve 420 suggests the number of the DNA target region is not exponentially doubled and sample 142 is collected from a patient who is not infected by the virus.

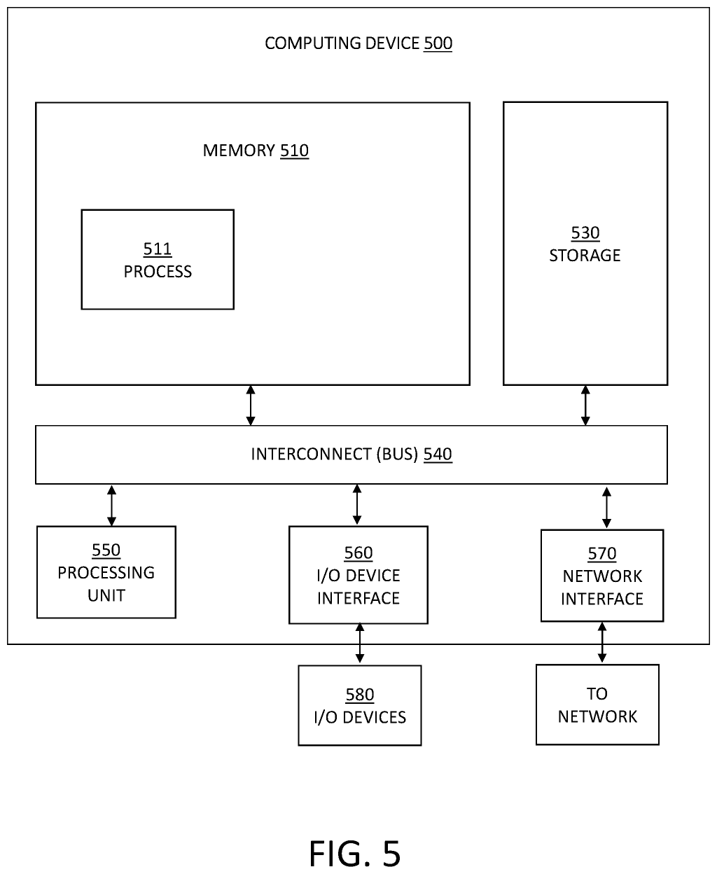

FIG. 5 is an illustration of computing device 500 configured to perform various embodiments of the present disclosure. Computing device 500 may correspond to computing device 160 of FIG. 1 in some embodiments. It is noted that the computing device described herein is illustrative and that any other technically feasible configurations fall within the scope of the present disclosure.

As shown, computing device 500 includes, without limitation, an interconnect (bus) 540 that connects a processing unit 550, an input/output (I/O) device interface 560 coupled to input/output (I/O) devices 580, memory 510, a storage 530, and a network interface 570. Processing unit 550 may be any suitable processor implemented as a central processing unit (CPU), a graphics processing unit (GPU), an application-specific integrated circuit (ASIC), a field programmable gate array (FPGA), any other type of processing unit, or a combination of different processing units, such as a CPU configured to operate in conjunction with a GPU or digital signal processor (DSP). In general, processing unit 550 may be any technically feasible hardware unit capable of processing data and/or executing software applications, including a process 511 consistent with method 200.

I/O devices 580 may include devices capable of providing input, such as a keyboard, a mouse, a touch-sensitive screen, and so forth, as well as devices capable of providing output, such as a display device and the like. Additionally, I/O devices 1480 may include devices capable of both receiving input and providing output, such as a touchscreen, a universal serial bus (USB) port, and so forth. I/O devices 580 may be configured to receive various types of input from an end-user of computing device 500, and to also provide various types of output to the end-user of computing device 500, such as displayed digital images or digital videos. In some embodiments, one or more of I/O devices 580 are configured to couple computing device 500 to a network.

Memory 510 may include a random access memory (RAM) module, a flash memory unit, or any other type of memory unit or combination thereof. Processing unit 550, I/O device interface 560, and network interface 570 are configured to read data from and write data to memory 510. Memory 510 includes various software programs that can be executed by processor 550.

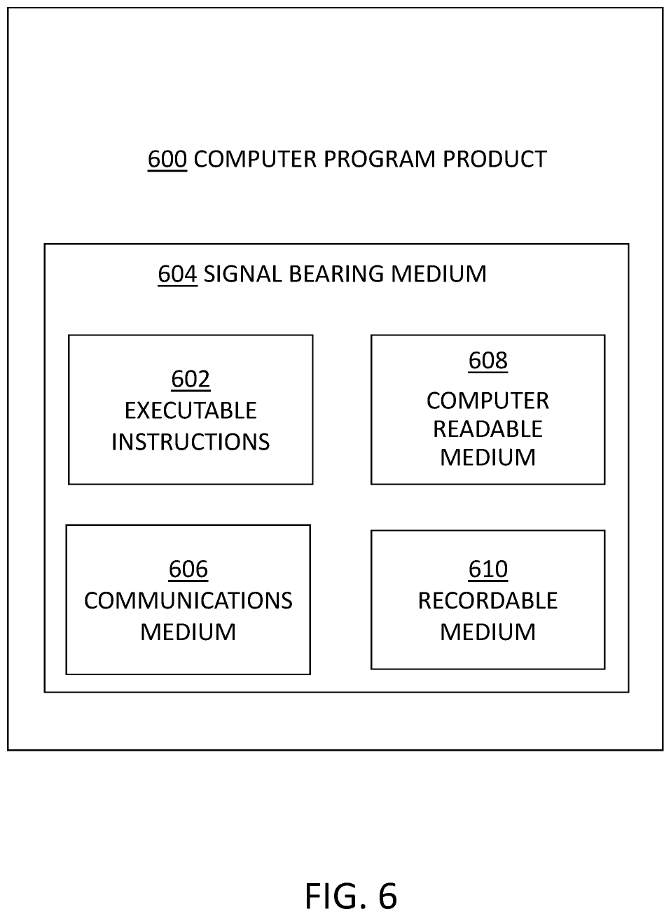

FIG. 6 is a block diagram of an illustrative embodiment of a computer program product 600 for implementing various embodiments of the present disclosure. Computer program product 600 may include a signal bearing medium 604. Signal bearing medium 604 may include one or more sets of executable instructions 602 that, when executed by, for example, a processor of a computing device, may provide at least the functionality described above with respect to method 200.

In some implementations, signal bearing medium 604 may encompass a non-transitory computer readable medium 608, such as, but not limited to, a hard disk drive, a Compact Disc (CD), a Digital Video Disk (DVD), a digital tape, memory, solid state drive, etc. In some implementations, signal bearing medium 604 may encompass a recordable medium 610, such as, but not limited to, memory, read/write (R/W) CDs, R/W DVDs, solid state drive, etc. In some implementations, signal bearing medium 604 may encompass a communications medium 606, such as, but not limited to, a digital and/or an analog communication medium (e.g., a fiber optic cable, a waveguide, a wired communications link, a wireless communication link, etc.). Computer program product 600 may be recorded on non-transitory computer readable medium 608 or another similar recordable medium 610.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A system configured to perform quantitative polymerase chain reaction (qPCR), comprising:
a first lighting subsystem including a first light source, wherein the first light source is configured to illuminate a qPCR testing solution including a sample, a fluorescence dye and a nanoparticle in a tube with a first light to increase a temperature of the qPCR testing solution; and
a second lighting subsystem including a light detector configured to detect an amount of fluorescence emitted by the fluorescence dye in a first thermal cycle of the qPCR,
wherein the first lighting subsystem is configured to turn off the first light source for a period of time in any thermal cycle of the qPCR after the first thermal cycle based on one or more heat transmission parameters which are determined based on the amount of fluorescence.

2. The system of claim 1, wherein the second lighting subsystem further includes a second light source configured to illuminate the qPCR testing solution with a second light.

3. The system of claim 2, further comprising a dichroic mirror configured to reflect the second light from the second light source to the qPCR testing solution.

4. The system of claim 3, wherein the dichroic mirror is configured to allow the fluorescence emitted by the fluorescence dye pass through the dichroic mirror.

5. The system of claim 2, further comprising a first color filter configured to filter out lights having a wavelength of the fluorescence, wherein the first color filter is on a first light path from second light source to the qPCR testing solution.

6. The system of claim 5, further comprising a second color filter configured to filter out lights having a wavelength of the second light source, wherein the second color filter is on a second light path from the qPCR testing solution to the light detector.

7. The system of claim 1, further comprising a cooling subsystem configured to decrease the temperature of the sample.

8. A method for performing quantitative polymerase chain reaction (qPCR), the method comprising:
in a first thermal cycle of performing the qPCR:
determining one or more heat transmission parameters based on an amount of fluorescence emitted by a fluorescence dye included in a qPCR testing solution in a tube; and
determining a first time duration of turning on a first light source configured to illuminate the qPCR testing solution further including a nanoparticle and a sample with a first light, a second time duration of turning off the first light source, a first power of the first light source and a second power of the first light source based on the one or more heat transmission parameters.

9. The method of claim 8, further comprising:
in a thermal cycle of performing the qPCR after the first thermal cycle:
turning on the first light source to illuminate the qPCR testing solution with the first light source for the first time duration;
adjusting a power of the first light source to the first power;
turning off the first light source for the second time duration; and
adjusting the power of the first light source to the second power.

10. The method of claim 9, further comprising increasing a temperature of the sample from a lower predefined temperature to perform the qPCR in the thermal cycle to a higher predefined temperature to preform the qPCR in the thermal cycle by turning on the first light source to illuminate the qPCR testing solution with the first light source for the first time duration.

11. The method of claim 9, further comprising maintaining a temperature of the sample at a higher predefined temperature to preform the qPCR in the thermal cycle by adjusting the power of the first light source to the first power.

12. The method of claim 9, further comprising decreasing a temperature of the sample from a higher predefined temperature to perform the qPCR in the thermal cycle to a lower predefined temperature to preform the qPCR in the thermal cycle by turning off the first light source for the second time duration.

13. The method of claim 9, further comprising maintaining a temperature of the sample at a lower predefined temperature to preform the qPCR in the thermal cycle by adjusting the power of the first light source to the second power.

14. The method of claim 9, further comprising repeating the turning on the first light source to illuminate the qPCR testing solution with the first light source for the first time duration; the adjusting a power of the first light source to the first power; the turning off the first light source for the second time duration; and the adjusting the power of the first light source to the second power for a plurality of thermal cycles of performing the qPCR after the first thermal cycle.

15. A non-transitory computer readable medium that includes a set of instructions which, in response to execution by a processor of a computing device, cause the computing device to perform quantitative polymerase chain reaction (qPCR), wherein the method comprises:
in a first thermal cycle of performing the qPCR:
determining one or more heat transmission parameters based on an amount of fluorescence emitted by a fluorescence dye included in a qPCR testing solution in a tube; and
determining a first time duration of turning on a first light source configured to illuminate the qPCR testing solution further including a nanoparticle and a sample with a first light, a second time duration of turning off the first light source, a first power of the first light source and a second power of the first light source based on the one or more heat transmission parameters.

16. The non-transitory computer readable medium of claim 15, wherein the method further comprises:
in a thermal cycle of performing the qPCR after the first thermal cycle:
turning on the first light source to illuminate the qPCR testing solution with the first light source for the first time duration;
adjusting a power of the first light source to the first power;
turning off the first light source for the second time duration; and
adjusting the power of the first light source to the second power.

17. The non-transitory computer readable medium of claim 16, wherein the method further comprises increasing a temperature of the sample from a lower predefined temperature to perform the qPCR in the thermal cycle to a higher predefined temperature to preform the qPCR in the thermal cycle by turning on the first light source to illuminate the qPCR testing solution with the first light source for the first time duration.

18. The non-transitory computer readable medium of claim 16, wherein the method further comprises maintaining a temperature of the sample at a higher predefined temperature to preform the qPCR in the thermal cycle based on adjusting the power of the first light source to the first power.

19. The non-transitory computer readable medium of claim 16, wherein the method further comprises decreasing a temperature of the sample from a higher predefined temperature to perform the qPCR in the thermal cycle to a lower predefined temperature to preform the qPCR in the thermal cycle by turning off the first light source for the second time duration.

20. The non-transitory computer readable medium of claim 16, wherein the method further comprises maintaining a temperature of the sample included in the qPCR testing solution at a lower predefined temperature to preform the qPCR in the thermal cycle by adjusting the power of the first light source to the second power.
